(19) United States
(12) Patent Application Publication
(10) Pub. No.: US 2004/0235802 A1
(43) Pub. Date: Nov. 25, 2004

(54) Combinations comprising cox-2-inhibitors and aspirin

(76) Inventor: Alberto Gimona, Basel (CH)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/487,759

(22) PCT Filed: Oct. 10, 2002
(86) PCT No.: PCT/EP02/11380

(30) Foreign Application Priority Data
Oct. 11, 2001 (GB) 0124459.9

(51) Int. Cl. A61K 31/415; A61K 31/60

(52) U.S. Cl. 514/165; 514/406

(57) ABSTRACT

A pharmaceutical compsition is provided for treatment of conditions in mammals which are responsitive to COX-2 inhibition which comprises in combination a COX-2 inhibitor and low-dosa aspirin for simultaneous, sequential or separate use.

This invention relates to pharmaceutical compositions and uses, in particular to pharmaceutical compositions for use in the selective inhibition of COX-2 activity and for treating conditions in mammals which are responsive to COX-2 inhibition.

It has been proposed to treat a condition selected from the group consisting of acute coronary ischemic syndrome, thrombosis, thromboembolism, thrombotic occlusion and reocclusion, transient ischemic attack, and first or subsequent thrombotic stroke, in a patient having the condition, comprising administering to the patient a therapeutically effective amount of an antiplatelet agent in combination with a therapeutically effective amount of a COX-2 inhibitor (U.S. Pat. No. 6,136,804; Merck). This combination therapy is stated to provide enhanced treatment options as compared to administration of either the antiplatelet agent or the COX-2 inhibitor alone. Aspirin is identified as an antiplatelet agent that may be used in this combination therapy and recommended for use at dosages generally in the range from 75 mg up to about 325 mg per day. It has now been found, in accordance with the present invention, that diseases involving platelet aggregation, such as those identified above, may be treated or avoided during treatment with a COX-2 inhibitor if the COX-2 inhibitor is administered in combination with aspirin at dosages lower than hitherto used; and furthermore that particular advantageous results are obtained if a 5-alkyl-2-arylaminophenylacetic acid derivative COX-2 inhibitor is used in combination with aspirin as antiplatelet inhibitor.

Accordingly the present invention provides a pharmaceutical composition for treatment of conditions in mammals which are responsive to COX-2 inhibition which comprises in combination an effective amount of a COX-2 inhibitor and low-dose aspirin, for simultaneous, sequential or separate use.

Further the invention provides the use of a COX-2 inhibitor for the preparation of a medicament, for use in combination with low-dose aspirin for treatment of conditions in mammals which are responsive to COX-2 inhibition.

In a further embodiment the invention provides a method of treating a patient suffering from a condition which is responsive to COX-2 inhibition comprising administering to the patient an effective amount of a COX-2 inhibitor in combination with low-dose aspirin.

Yet further the invention provides use of low-dose aspirin to treat acute coronary ischemic syndrome, thrombosis, thromboembolism, thrombotic occlusion and reocclusion, transient ischemic attack, myocardial infarction, and first or subsequent thrombotic stroke, in a patient having the condition, when the low-dose aspirin is administered in combination with an effective amount of a COX-2 inhibitor. Advantageously low dose aspirin is administered together with the COX-2 inhibitor for cardio-protection, e.g. in view of the anti-platelet aggregation activity of aspirin.

In the present description the term “treatment” includes both prophylactic or preventative treatment as well as curative or disease modifying treatment, including treatment of patients at risk of contracting the disease or suspected to have contracted the disease as well as ill patients. In preferred embodiments of the invention “treatment” comprises primary or secondary prevention of cardiovascular disease.

The invention is generally applicable to the treatment of conditions in mammals which are responsive to COX-2 inhibition. For instance, for the treatment of cyclooxygenase dependent disorders in mammals, including inflammation, pyresis, pain, osteoarthritis, rheumatoid arthritis, migraine headache, neurodegenerative diseases (such as multiple sclerosis), Alzheimer's disease, osteoporosis, asthma, lupus and psoriasis. Moreover, COX-2 inhibitors are further useful for the treatment of neoplasia particularly neoplasia that produce prostaglandins or express cyclooxygenase, including both benign and cancerous tumors, growths and polyps. COX-2 inhibitors may be employed for the treatment of any neoplasia as for example as recited in International Patent Application Publication No. WO 98116227, published 23 Apr. 1998, in particular epithelium cell-derived neoplasia. COX-2 inhibitors are in particular useful for the treatment of liver, bladder, pancreas, ovarian, prostate, cervical, lung and breast cancer and, especially gastrointestinal cancer, for example cancer of the colon, and skin cancer, for example squamus cell or basal cell cancers and melanoma.

The compositions, uses and methods of the present invention represent an improvement to existing therapy of conditions in mammals which are responsive to COX-2 inhibition.

In the present description the term “low-dose aspirin” means an aspirin dose of less than 75 mg per day, typically a dose in the range from about 70 mg down to about 10 mg or less (e.g. at least about 5 mg) per day. Preferred low-dose aspirin dosages are in the range from about 20 mg up to about 60 mg per day, more preferably from about 30 mg up to about 50 mg per day.

The COX-2 inhibitors used in the pharmaceutical compositions and treatment methods of the present invention are typically those which have an IC₅₀ for COX-2 inhibition less than about 2 μM and an IC₅₀ for COX-1 inhibition greater than about 5 μM, e.g. when measured in the assays described by Brideau et al.in Inflamm. Res. 45:68-74 (1996). Preferably the COX-2 inhibitor has a selectivity ratio of at least 10, more preferably at least 40, for COX-2 inhibition over COX-1 inhibition.

Thus, for example, suitable COX-2 inhibitors for use in the invention may include any of the COX-2 inhibitors identified in U.S. Pat. No. 6,136,804; in particular the following compounds or a pharmaceutically acceptable salt thereof, or any hydrate thereof: rofecoxib, etoricoxib, celecoxib, valdecoxib, parecoxib, or a 5-alkyl-2-arylaminophenylacetic acid derivative COX-2 inhibitor, e.g. of formula I as defined below.

In a particular embodiment a COX-2 inhibitor for use in the present invention comprises a compound of formula I

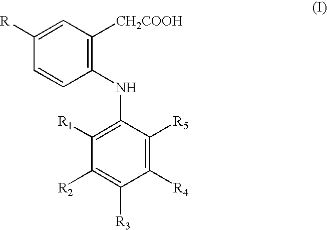

wherein R is methyl or ethyl;

R₁ is chloro or fluoro;

R₂ is hydrogen or fluoro;

R₃ is hydrogen, fluoro, chloro, methyl, ethyl methoxy, ethoxy or hydroxy;

R₄ is hydrogen or fluoro; and

R₅ is chloro, fluoro, trifluoromethyl or methyl;

pharmaceutically acceptable salts thereof, and

pharmaceutically acceptable prodrug esters thereof.

Particular compounds of formula I are those wherein R is methyl or ethyl; R₁ is chloro or fluoro; R₂ is hydrogen; R₃ is hydrogen, fluoro, chloro, methyl or hydroxy, R₄ is hydrogen; and R₅ is chloro, fluoro or methyl; pharmaceutically acceptable salts thereof; and pharmaceutically acceptable esters thereof.

A preferred embodiment relates to the compounds of formula I wherein R is methyl or ethyl; R₁ is fluoro; R₂ is hydrogen; R₃ is hydrogen, fluoro or hydroxy; R₄ is hydrogen; and R₅ is chloro; pharmaceutically acceptable salts thereof; and pharmaceutically acceptable prodrug esters thereof.

Another preferred embodiment of the invention relates to compounds of formula I wherein R is ethyl or methyl; R₁ is fluoro; R₂ is hydrogen or fluoro; R₃ is hydrogen, fluoro, ethoxy or hydroxy; R₄ is hydrogen or fluoro; and R₅ is chloro, fluoro or methyl; pharmaceutically acceptable salts thereof, and pharmaceutically acceptable prodrug esters thereof.

Further preferred are said compounds wherein R is methyl or ethyl; R₁ is fluoro; R₂-R₄ are hydrogen or fluoro; and R₅ is chloro or fluoro; pharmaceutically acceptable salts thereof, and pharmaceutically acceptable prodrug esters thereof.

A further embodiment of the invention relates to the compounds of formula I wherein R is methyl or ethyl; R₁ is fluoro; R₂ is fluoro; R₃ is hydrogen, ethoxy or hydroxy, R₄ is fluoro; and R₅ is fluoro; pharmaceutically acceptable salts thereof; and pharmaceutically acceptable prodrug esters thereof.

Another preferred embodiment of the invention relates to the compounds of formula I wherein R is methyl; R₁ is fluoro; R₂ is hydrogen; R₃ is hydrogen or fluoro; R₄ is hydrogen; and R₅ is chloro; pharmaceutically acceptable salts thereof; and pharmaceutically acceptable prodrug esters thereof.

Particular embodiments of the invention relate to compounds of formula I

(a) wherein R is methyl; R₁ is fluoro; R₂ is hydrogen; R₃ is hydrogen; R₄ is hydrogen; and R₅ is chloro; pharmaceutically acceptable salts thereof; and pharmaceutically acceptable prodrug esters thereof,

(b) wherein R is methyl; R₁ is fluoro; R₂ is hydrogen; R₃ is fluoro; R₄ is hydrogen; and R₅ is chloro; pharmaceutically acceptable salts thereof; and pharmaceutically acceptable prodrug esters thereof,

(c) wherein R is ethyl; R₁ is fluoro; R₂ is fluoro; R₃ is hydrogen; R₄ is fluoro; and R₅ is fluoro; pharmaceutically acceptable salts thereof; and pharmaceutically acceptable prodrug esters thereof; and

(d) wherein R is ethyl; R₁ is chloro; R₂ is hydrogen; R₃ is chloro; R₄ is hydrogen; and R₅ is methyl; pharmaceutically acceptable salts thereof; and pharmaceutically acceptable prodrug esters thereof.

The general definitions used herein have the following meaning within the scope of the present invention

The compounds of formula I are UV absorbers and are useful for blocking or absorbing UV radiation; for instance, for the treatment and prevention of sunburn, e.g. in suntan products

The compounds of formula I may also be used in ocular applications which include the treatment of ocular disorders, in particular of ocular inflammatory disorders, of ocular pain including pain associated with ocular surgery such as PRK or cataract surgery, of ocular allergy, of photophobia of various etiology, of elevated intraocular pressure (in glaucoma) by inhibiting the production of trabecular meshwork inducible glucocorticoid response (TIGR) protein, and of dry eye disease.

In a second aspect the invention also provides a pharmaceutical composition for treatment of conditions in mammals which are responsive to COX-2 inhibition which comprises in combination an effective amount of a compound of formula I or a pharmaceutically acceptable salt or prodrug thereof and an effective amount of aspirin, for simultaneous, sequential or separate use.

Further the invention provides the use of a compound of formula I or a pharmaceutically acceptable salt or prodrug thereof for the preparation of a medicament, for use in combination with an effective amount of aspirin for treatment of conditions in mammals which are responsive to COX-2 inhibition.

In a yet further embodiment of this second aspect the invention provides a method of treating a patient suffering from a condition which is responsive to COX-2 inhibition comprising administering to the patient an effective amount of a compound of formula I or a pharmaceutically acceptable salt or prodrug thereof in combination with an effective amount of aspirin.

The “effective amount of aspirin” for use in this second aspect of the invention includes those amounts commonly known and used by physicians when using aspirin as an anti-platelet agent. Conveniently the “effective amount of aspirin” is generally in the range from about 10 mg to about 400 mg, more usually from about 75 mg to about 325 mg per day. For example, the composition of this second aspect may contain 75 mg, 80 mg, 160 mg, 250 mg or 325 mg of aspirin.

Pharmaceutically acceptable salts of the compound of formula I are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

The Agents of the Invention (a the COX-2 inhibitor and the low-dose apirin or b. the compound of formula I or pharmaceuticaly acceptable salt or prodrug thereof and effective amount of aspirin) are preferably used in the form of pharmaceutical preparations that contain the relevant therapeutically effective amount of of each active ingredient (either separately or in combination) optionally together with or in admixture with inorganic or organic, solid or liquid, pharmaceutically acceptable carriers which are suitable for administration. The COX-2 inhibitor and aspirin active ingredients may be present in the same pharmaceutical compositions, though are preferably in separate pharmaceutical compositions. Thus the active ingredients may be administered at the same time (e.g. simultaneously) or at different times (e.g. sequentially) and over different periods of time, which may be separate from one another or overlapping.

The pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the pharmaceutical compositions are adapted to oral or parenteral (especially oral) administration. Intravenous and oral, first and foremost oral, adminstration is considered to be of particular importance. Preferably both the COX-2 inhibitor and aspirin active ingredient are in oral form.

The particular mode of administration and the dosage may be selected by the attending physician taking into account the particulars of the patient, especially age, weight, life style, activity level, etc.

The dosage of the Agents of the Invention may depend on various factors, such as effectiveness and duration of action of the active ingredient, mode of administration, warm-blooded species, and/or sex, age, weight and individual condition of the warm-blooded animal.

More particularly, the pharmaceutical compositions comprise an effective cyclooxygenase-2 inhibiting amount of COX-2 inhibitor or compound of formula I which is substantially free of cyclooxygenase-1 inhibiting activity and of side effects attributed thereto.

The pharmacologically active compounds of the invention are useful in the manufacture of pharmaceutical compositions comprising an effective amount thereof in conjunction or admixture with excipients or carriers suitable for either enteral or parenteral application. Preferred are tablets and gelatin capsules comprising the active ingredient together with a) diluents, e.g. lactose, dextrose, sucrose, mannitol, sorbitol, cellulose and/or glycine; b) lubricants, e.g. silica, talcum, stearic acid, its magnesium or calcium salt and/or polyethyleneglycol; for tablets also c) binders e.g. magnesium aluminum silicate, starch paste, gelatin, tragacanth, methylcellulose, sodium carboxymethylcellulose and or polyvinylpyrrolidone; if desired d) disintegrants, e.g. starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 75%, preferably about 1 to 50%, of the active ingredient.

Tablets may be either film coated or enteric coated according to methods known in the art.

Suitable formulations for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable formulations for topical application, e.g. to the skin and eyes, include aqueous solutions, suspensions, ointments, creams, gels or sprayable formulations, for example, for delivery by aerosol or the like. Such topical delivery systems will in particular be appropriate for dermal application, e.g. for the treatment of skin cancer, for example, for prophylactic use in sun creams, lotions sprays and the like. In this regard it is noted that compounds of formula I are capable of absorbing UV rays in the range of 290-320 nm while allowing passage of tanning rays at higher wavelenghts. They are thus particularly suited for use in topical, including cosmetic formulations as aforesaid well-known in the art. Such may contain solubilizers, stabilizers, tonicity enhancing agents, buffers and preservatives. Formulations suitable for topical application can be prepared e.g. as described in U.S. Pat. No. 4,784,808. Formulations for ocular administration can be prepared e.g. as described in U.S. Pat. Nos. 4,829,088 and 4,960,799.

The dosage of COX-2 inhibitor administered is dependent on the species of warm-blooded animal (mammal), the body weight, age and individual condition, and on the form of administration. A unit dosage for oral administration to a mammal of about 50 to 70 kg may contain between about 5 and 1000 mg, e.g. from 50-800 mg, preferably 100-500 mg of the active ingredient.

COX-2 inhibitor formulations in single dose unit form contain preferably from about 1% to about 90%, and formulations not in single dose unit form contain preferably from about 0.1% to about 20%, of the active ingredient. Single dose unit forms such as capsules, tablets or dragées contain e.g. from about 1 mg to about 1000 mg of the active ingredient.

COX-2 inhibitor pharmaceutical preparations for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

The following examples are intended to illustrate the invention and are not to be construed as being limitations thereon.

### FORMULATION EXAMPLES

### Example 1

Wet granulated tablet composition

| Amount per tablet | Ingredient |
|---|---|
| 25 mg | COX-2 inhibitor |
| 79.7 mg | Microcrystalline cellulose |
| 79.7 mg | Lactose monohydrate |
| 6 mg | Hydroxypropyl cellulose |
| 8 mg | Croscarmellose sodium |
| 0.6 mg | Iron oxide |
| 1 mg | Magnesium stearate |

Tablet dose strengths of between 5 and 125 mg can be accomodated by varying total weight, and the ratio of the first three ingredients. Generally it is preferable to maintain a 1:1 ratio for microcrystalline cellulose: lactose monohydrate.

### Example 2

Wet granulated tablet composition

| Amount per tablet | Ingredient |
|---|---|
| 12.5 mg | COX-2 inhibitor |
| 86 mg | Microcrystalline cellulose |
| 86 mg | Lactose monohydrate |
| 6 mg | Hydroxypropyl cellulose |
| 8 mg | Croscarmellose sodium |
| 0.6 mg | Iron oxide |
| 1 mg | Magnesium stearate |

### Example 3

Wet granulated tablet composition

| Amount per tablet | Ingredient |
|---|---|
| 10 mg | COX-2 inhibitor |
| 87.2 mg | Microcrystalline cellulose |
| 87.2 mg | Lactose monohydrate |
| 6 mg | Hydroxypropyl cellulose |
| 8 mg | Croscarmellose sodium |
| 0.6 mg | Iron oxide |
| 1 mg | Magnesium stearate |

### Example 4

Wet granulated tablet composition

| Amount per tablet | Ingredient |
|---|---|
| 5 mg | COX-2 inhibitor |
| 89.7 mg | Microcrystalline cellulose |
| 89.7 mg | Lactose monohydrate |
| 6 mg | Hydroxypropyl cellulose |
| 8 mg | Croscarmellose sodium |
| 0.6 mg | Iron oxide |
| 1 mg | Magnesium stearate |

### Example 5

Directly compressed tablet composition

| Amount per tablet | Ingredient |
|---|---|
| 25 mg | COX-2 inhibitor |
| 106.9 mg | Microcrystalline cellulose |
| 106.9 mg | Lactose anhydrate |
| 7.5 mg | Croscarmellose sodium |
| 3.7 mg | Magnesium stearate |

Tablet dose strengths of between 5 and 125 mg can be accomodated by varying total tablet weight, and the ratio of the first three ingredients. Generally it is preferable to maintain a 1:1 ratio for microcrystalline cellulose:lactose monohydrate.

### Example 6

Directly compressed tablet composition

| Amount per tablet | Ingredient |
|---|---|
| 12.5 mg | COX-2 inhibitor |
| 113.2 mg | Microcrystalline cellulose |
| 113.2 mg | Lactose anhydrate |
| 7.5 mg | Croscarmellose sodium |
| 3.7 mg | Magnesium stearate |

### Example 7

Directly compressed tablet composition

| Amount per tablet | Ingredient |
|---|---|
| 10 mg | COX-2 inhibitor |
| 42.5 mg | Microcrystalline cellulose |
| 42.5 mg | Lactose anhydrate |
| 4 mg | Croscarmellose sodium |
| 1 mg | Magnesium stearate |

### Example 8

Directly compressed tablet composition

| Amount per tablet | Ingredient |
|---|---|
| 5 mg | COX-2 inhibitor |
| 45 mg | Microcrystalline cellulose |
| 45 mg | Lactose anhydrate |
| 4 mg | Croscarmellose sodium |
| 1 mg | Magnesium stearate |

### Example 9

Hard gelatine capsule composition

| Amount per capsule | | Ingredient |
|---|---|---|
| 25 | mg | COX-2 inhibitor |
| 37 | mg | Microcrystalline cellulose |
| 37 | mg | Lactose anhydrate |
| 1 | mg | Magnesium stearate |
| 1 | capsule | Hard gelatin capsule |

Capsule dose strengths of between 1 and 50 mg can be accomodated by varying total fill weight, and the ratio of the first three ingredients. Generally it is preferable to maintain a 1:1 ratio for microcrystallin cellulose:lactose monohydrate.

### Example 10

Oral solution

| Amount per 5 mL | | Ingredient |
|---|---|---|
| 50 | mg | COX-2 inhibitor |
| to 5 | mL | with Polyethylene oxide 400 |

### Example 11

Oral suspension

| Amount per 5 mL dose | Ingredient |
|---|---|
| 101 mg | COX-2 inhibitor |
| 150 mg | Polyvinylpyrrolidone |

Oral suspension

| Amount per 5 mL dose | | Ingredient |
|---|---|---|
| 2.5 | mg | Poly oxyethylene sorbitan monolaurate |
| 10 | mg | Benzoic acid |
| to 5 | mL | with sorbitol solution (70%) |

Suspension dose strengths of between 1 and 50 mg/5 ml can be accomodated by varying the ratio of the first two ingredients.

### Example 12

Intravenous infusion

| Amount per 200 mL dose | | Ingredient |
|---|---|---|
| 1 | mg | COX-2 inhibitor |
| 0.2 | mg | Polyethylene oxide 400 |
| 1.8 | mg | Sodium chloride |
| to 200 | mL | Purified water |

### Example 13

Combination Tablet Preparation

Tablets containing 25.0, 50.0 and 100.0 mg, respectively, of a GP IIb/IIIa receptor antagonist and 25 mg COX-2 Inhibitor are prepared as illustrated below:

Table for doses containing from 25-200 mg of aspirin and 25 mg COX-2 inhibitor

| | Amount mg | | |
|---|---|---|---|
| aspirin | 25.0 | 80.0 | 200.0 |
| COX-2 inhibitor | 25.0 | 25.0 | 25.0 |
| Microcrystalline cellulose | 37.25 | 100.0 | 175.0 |
| Modified food corn starch | 37.25 | 4.25 | 8.5 |
| Magnesium stearate | 0.50 | 0.75 | 1.5 |

Both active compounds, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of GP IIb/IIIa receptor antagonist per tablet, and 25 mg COX-2 inhibitor, per tablet.

### Example 14

TABLE 1

| | Amount per 200 mg |
|---|---|
| Ingredient | tablet batch (kg) |
| Core | |
| Granulation | |
| 5-methyl-2-(2′-chloro-6′- | 50** |
| fluoroanilino)phenylacetic acid drug | |
| substance | |
| Microcrystalline cellulose, NF (PH | 12.85 |
| 101) | |
| Lactose monohydrate, NF | 11.65 |
| Croscarmellose sodium, NF | 1 |
| Povidone, USP | 4 |
| Titanium dioxide, USP | 2 |
| Water, purified***, USP | 20.375 |
| Extra-granular Phase | |
| Microcrystalline cellulose, NF (PH | 13 |
| 102) | |
| Croscarmellose sodium, NF | 3 |
| Titanium dioxide, USP | 2 |
| Magnesium stearate, NF | 0.5 |
| Coating | |
| Opadry white | 2.801**** |
| Opadry yellow | 2.0**** |
| Opadry red | 0.4**** |
| Opadry black | 0.0504**** |
| Water, purified***, USP | 29.758**** |

**The weight of drug substance is taken with reference to the dried substance (100 percent) on the basis of the assay value (factorization). The difference in weight is adjusted by the amount of microcrystalline cellulose used.
***Removed during processing.
****Includes a 50% excess for loss during the coating process.

Table 1, above, sets out the formula for a batch of approximately 250,000 immediate release film-coated tablets of 5-methyl-2-(2′-chloro-6′-fluoroanilino)-phenylacetic acid. To make the tablets, titanium dioxide is dispersed in water, followed by the addition of povidone and mixing for 20 minutes to make a povidone/titanium dioxide suspension. The drug substance, lactose, microcrystalline cellulose, and croscarmellose are mixed in a high shear mixer (e.g., a Collette Gral) for 5 minutes to form a drug mixture. The drug mixture is granulated in the high shear mixer with the povidone/titanium dioxide suspension. The suspension is pumped at a rate of 3 kg/min into the drug mixture. The resulting mixture is mixed an additional 90 seconds after all the suspension is added. The wet granulation is dried in a fluid bed dryer, using an inlet air temperature of 50° C. The residual water target is 3.5% (with a permissible range of 2.5-4.5%). The dried granulation is passed through a screen using a mill (oscillator) and a 30 mesh screen. The previous steps are repeated to make a second granulation.

The extra-granular phase titanium dioxide is passed through a 60 mesh hand screen. The dry granulations are mixed with the extra-granular phase microcrystalline cellulose, croscarmellose sodium and titanium dioxide in a twin shell mixer for 300 revolutions to form a penultimate mixture. Magnesium stearate is passed through a 60 mesh hand screen and is mixed with the penultimate mixture in a twin shell mixer for 50 revolutions to form a tableting mixture. The tableting mixture is pressed into tablets using a tablet press and oval punches.

The coating powders (Opadry) are mixed with purified water to make a 15% w/w coating suspension. The tablets are film coated with the coating suspension in a coating pan using 60° C. to 75° C. inlet air temperature.

Table 2 sets out the contents of a 200 mg 5-methyl-2-(2′-chloro-6′-fluoroanilino)phenylacetic acid film-coated tablet.

TABLE 2

| | Theoretical | |
|---|---|---|
| Ingredient | amount [mg] | Function |
| Core | | |
| 5-methyl-2-(2′-chloro-6′- | 200 | Active substance |
| fluoroanilino)phenylacetic acid | | |
| drug substance | | |
| Microcrystalline cellulose (PH | 51.4 | Filler |
| 101) | | |
| Lactose | 46.6 | Filler |
| Povidone | 16 | Binder |
| Titanium dioxide | 8 | Color |
| Croscarmellose sodium | 4 | Disintegrant |
| Water, purified* | Q.S. | Granulating |
| | | liquid |
| Extragranular phase | | |
| Microcrystalline cellulose (PH | 52 | Filler |
| 102) | | |
| Croscarmellose sodium | 12 | Disintegrant |
| Titanium dioxide | 8 | Color |
| Magnesium stearate | 2 | Lubricant |
| Core weight | 400 | |
| Coating | | |
| Opadry white (00F18296) | 7.4676 | Color |
| Opadry yellow (00F12951) | 5.3312 | Color |
| Opadry red (00F15613) | 1.0668 | Color |
| Opadry black (00F17713) | 0.1344 | Color |
| Water, purified* | Q.S. | Coating solvent |
| Total weight | 414 | |

*removed during processing

In addition, the tablet formulations may contain 5-methyl-2-(2′-chloro-6′-fluoroanilino)benzyl alcohol and/or 5-methyl-2-(2′-chloro-6′-fluoroanilino)benzoic acid in an amount between about 0.01 and 2% by weight, more specifically between about 0.1 and 1.

### Clinical Study

The following clinical study is carried out using 5-methyl-2-(2′-chloro-6′-fluoroanilino phenylacetic acid (COX 189) as selective COX-2 inhibitor.

Study no/phase COX189 2408 (a compound of formula I)—GI outcome endpoint (Worlwide)

The study is known as Lumiracoxib TARGET (Therapeutic COXI 89 Arthritis Research & Gastrointestinal Event Trial) and consists of two parts: The study, CCOX189 0117 (or COX189 TARGET I) is the first part; the study CCOX189 2332 (or COX189 TARGET II) is the second part.

The design of both studies is identical the only difference is the comparator: naproxen 500 mg bid for study CCOX189 0117 and ibuprofen 800 mg bid for study CCOX189A2332. The results of both studies will be pooled.

Status

Title A international multicenter, stratified, randomized, double-blind, double-dummy, parallel-group, 52-week gastrointestinal safety study to demonstrate that COX189 (400 mg od) reduces the risk of developing complicated ulcers as compared to NSAIDS (naproxen, 500 mg bid and ibuprofen 800 mg tid) in osteoarthritis.

Objectives

Primary COX189 TARGET I and II:

1) An at least a 50% decrease of complicated ulcers (perforations, obstructions and bleedings) with COX189 as compared to NSAIDs (naproxen 500 mg bid and ibuprofen 800 mg tid) in patients not taking low-dose of aspirin.

2) An at least a 44% decrease of complicated ulcers (perforations, obstructions and bleedings) with COX189 as compared to NSAIDs (naproxen 500 mg bid and ibuprofen 800 mg tid) in the overall patient population.

Secondary COX189 TARGET I and II:

1) Assess cardiovascular and renal safety as well as the overall safety and tolerability profile of COX189 as compared to NSAIDs (naproxen 500 mg bid and ibuprofen 800 mg tid) in the overall patient population,

2) Assess the efficacy of COX189 as compared to NSAIDs (naproxen 500 mg bid and ibuprofen 800 mg tid) in the overall patient population.

Design PG Parallel Group, AC Active Comparator Controlled, MC Multi Centre, OP Out Patient, RS Randomized stratified,; DBDD Double Blind Double Dummy

Incl./Excl. Inclusion Criteria:

Criteria

Age: 50 y and above (no upper limit).

For OA patients: Patients with primary OA in any of the following joints with symptoms for at least 3 months are eligible: hip, knee, or hand according to ACR criteria, spine, cervical or lumbar (confirmed by X-ray; with absence of radicular symptoms).

H. pylori status: positive or negative eligible, serology testing at entry, investigators will remain blinded to the H. pylori status up to the end of the study.

Taking or requiring aspirin (75 mg-100 mg) for primary or secondary cardiovascular prevention. Patients should be on stable dose three months prior to enter the study.

With a baseline pain assessment (Likert scale) in the affected joint (referred to as the target joint which is the most affected joint: most painful) of moderate, severe or extreme.

Requiring NSAIDS therapy for treatment of OA regardless of whether or not they are currently receiving treatment. Patients will be expected to require NSAID treatment for at least 12 months.

Patients who were included in any previous COX189 trial are allowed to enter this study (patients can participate only in TARGET I or II).

Exclusion Criteria:

The following patients will be excluded:

The following patients will be excluded:

With the following secondary arthritis conditions: septic arthritis, inflammatory joint disease, gout, recurrent episodes of pseudogout, Paget's disease of bone, articular fracture, ochronosis, acromegaly, hemochromatosis, Wilson's disease, primary osteochondromatosis, heritable disorders (e.g. hypermobility), collagen gene mutations

If condition is ACTIVE, then patient should be excluded

If patient has history of these conditions, then patient should only be excluded if the TARGET joint is affected

With other rheumatic diseases, including but not limited to uncontrolled gout (acute gouty arthritis within the last 3 months), recurrent episodes of pseudogout (chondrocalcinosis without symptoms of pseudogout is allowed), primary fibromyalgia (secondary fibromyalgia is allowed in joints other than the target joint if, in the opinion of the investigator it will not interfere with patient's pain assessment), systemic lupus erythematosus, ankylosing spondylitis, polymyositis or dermatomyositis, vasculitic syndromes, scleroderma, psoriasis arthritis, reactive arthritis, active rheumatic fever, Sjogren's syndrome, mixed connective tissue disease, Behcet's syndrome, and rheumatoid arthritis.

Active peptic ulcer disease within 30 days of study screening.

History of POBs.

Who have any previous gastric surgery (e.g. resection or vagotomy) with the exception of a simple suture of an ulcer.

With a history of inflammatory bowel disease (e.g. ulcerative colitis or Crohns disease), or upper GI tract malignancies. With complicated diverticulosis. With a history of bleeding diathesis.

With evidence of hepatic (ALT, AST>1.5×ULN, renal (serum creatinine>1.25×ULN), total bilirubin>1.2×ULN or blood coagulation disorders (i.e. hemophilia) or anemia (hemoglobin less than 20 g/L below the lower limit of normal).

Who are currently taking proton pump inhibitors or misoprostol. Patients may not be wash-out to enter the study.

Who are taking sucralfate. Patients may be washed out to enter the study. The washout period must be at least one month prior to screening.

Who are currently on and high dose of H2 receptor antagonists (H2RA) e.g. ≧40 mg/day of famotidine or mid dose (e.g. ≧20 mg but <40 mg/day of famotidine). Low dose (e.g. <20 mg/day of famotidine or equivalent) may not be stopped to enter the trial, if it is at least 4 weeks prior to entry into the study.

Who have a cardiovascular history of:

coronary heart disease with ECG-evidence of silent myocardial ischemia.

congestive heart failure with symptoms at rest or with minimal activity (NYHA class III-IV).

unstable angina including:

crescendo angina: episodes of angina increasing in frequency

angina at rest and with minimal exertion including angina decubitus (without stimulation)

nocturnal angina (angina at night)

variant angina (Prinzmetal's angina).

Who had one of the following cardiovascular events occur within 6 months prior to screening:

myocardial infarction or stroke

underwent coronary artery bypass grafting or invasive coronary revascularization

new-onset angina

Who have ischemic cerebrovascular or cardiovascular disease and are not on low dose (75-100 mg/day) ASA (see Supplement 3b for a list of conditions).

With cardio rhythm abnormalities (atrial fibrillation, ventricular fibrillation, atrial flutter, ventricular tachycardia (detected on the baseline ECG).

Who are taking anticoagulants (e.g. warfarin, low-molecular weight heparin) and anti-platelet aggregation agents (except low-dose aspirin 75 mg-100 mg/day for cardioprotection).

Concomitant medications antiacids are allowed if taken no more than twice per week for calcium supplementation only but regular use must be stopped at screening. A same antiacid should be used per country.

Dose/regimen COX189: 400 mg od. Comparator: naproxen 500 mg bid and ibuprofen 800 mg tid.

Compliance: a patient will be considered compliant for study medication intake if he/she takes 75% of planned daily doses. This also means that a patient can in compliance with the protocol be off study drug for a maximum of 25% non consecutively of his/her time under study drug treatment.

An interactive voice response system (IVRS) will be used in all countries. It will work through toll free lines in most languages, with a help line (in some languages) as back-up. Both will be available 24 h a day, 7 days per week.

Treatment 1-week screening+52-week treatment+4-week follow-up duration The trial (COX189 TARGET I and II) will be stopped when 156 expected follow-up duration events are confirmed or when 52 weeks of treatment have been achieved for all patients in the combined studies.

ALL PATIENTS DROPPING WILL BE FOLLOWED-UP:

All patients will be contacted (e.g. by phone) 4 weeks after discontinuation for suspected serious gastrointestinal events and for suspected selected cardiovascular events (myocardial infarction, stroke and cardiovascular death).

| | |
|---|---|
| Recruitment | 39 weeks |
| period | |

| Total # of | # | # ran- | | # per |
|---|---|---|---|---|
| patients | screened | domized | # per arm | center |
| TARGET I & II | 22,408 | 18,672 | 9,336 COX189/ | |
| | | | 9,336 NSAID | |
| | | | (naproxen 3,168 | |
| | | | and | |
| | | | ibuprofen 3,168) | |
| TARGET I or | 11,204 | 9,336 | 4,668 | 16 |
| TARGET II | | | | |

| Key Dates | Protocol | FPFV | LPLV | Report |
|---|---|---|---|---|
| | 13 Jul. 2001 | 24 Nov. 2001 | December 2003 | May 2004 |

Assessments

Efficacy At all visits excepted Visit 1 and Visit 3

Patient (over the last week) and investigator assessment of disease activity using a Likert scale: very good, good, fair, poor, and very poor.

Patient pain assessment (over the last 24 h), using a Likert scale: none, mild, moderate, severe, extreme.

Safety (no scheduled endoscopy)

Assessments:

Complicated ulcers of the upper GI tract detected by clinical symptoms or finding (not by scheduled endoscopy)

Myocardial infraction, stroke and cardiovascular deaths

Central blinded review of clinical documentation for suspected cases of complicated ulcers and selected cardiovascular events (myocardial infraction, stroke and cardiovascular death) to categorize them according to pre-specified definitions. This review will be performed by an independent GI Safety Committee made up of 2 gastroenterologists and 1 epidemiologist and by a Cardiovascular and Cerebrovascular Safety Committee made up by 3 cardiologists and 2 neurologists. These committees will define procedures for assessment and assess all suspected cases from the Lumiracoxib TARGET study.

Vital signs, weight, ECG at baseline and end of study. All ECGs will be reviewed by central reading.

Serum chemistries, hematology, through a central laboratory for all countries, performed at all visits expected visits 2 and 6.

Pharmacogenetic assessments if patient consent.

PK None

PD None

List of Examinations

Prior concomitant medications, adverse events and serious adverse events will be collected by using the standard forms.

Visit Schedule:

Visit 1: Screening

Visit 2: Baseline (randomization)

Visit 3: 4 weeks/(plus or minus 4 days)

Visit 4: 13 weeks (plus or minus 2 weeks)

Visit 5: 20 weeks (plus or minus 2 weeks)

Visit 6: 26 weeks (plus or minus 2 weeks)

Visit 7: 39 weeks (plus or minus 2 weeks)

Visit 8: 52 weeks (plus or minus 2 weeks), or early discontinuation

Follow-up: by a phone call at 4 weeks after discontinuation for follow up on serious gastrointestinal events and for selected cardiovascular events (myocardial infarction, stroke and cardiovascular death).

About the Number of Patients:

Randomization will be stratified by age group (<65, 65 to 74, >74) and by use of low dose aspirin by an IVRS system (4,524 ASA users (ca. 24%) and 14,148 non ASA users (ca. 76%)).

Statistical Methods

The trial is designed to demonstrate that a significant difference in time-to-event curves on complicated ulcers of the upper gastrointestinal tract as compared to NSAIDs (naproxen and ibuprofen). For the primary endpoint an exponential maximum likelihood test of equality of time-to-event curves with one-sided significance level of 0.025 will be performed. A Cox proportional hazard model will be used to compare the relative risk between the two treatment groups. Covariates included in this model will be treatment group indicator and strata of age and prior history of POBs.

The Calculation of Sample Size is Based on the Following Assumptions:

A maximum individual follow-up time (treatment duration) is one year.

The drop out rate is expected to be equal across treatment arms. Overall, a drop out rate of 0.511/year (=probability of 60% that a patient is still in the trial after one year, if no event happened) is assumed. Other trials performed showed a higher drop out rate after start of individual treatment rather than during the conduct of the trial. Therefore, it is expected that 13% of enrolled patients will drop-out during the first month, 6% for the second month, 6% for the third month, 3% for months 4-6 and 1% for the last 6 months of individual treatment respectively.

For POBs in the group of patients which are not on low-dose aspirin and treated with NSAIDs, an overall incidence rate of 1.30% in both RA and OA patients (=hazard rate of 0.013%/year) is expected.

For POBs in the group of patients which are on low-dose aspirin and treated with NSAIDs, an overall incidence rate of 2.50% in both RA and OA patients (=hazard rate of 0.025%/year) is expected

For the primary analyses, the power will be 90% to detect a 50% reduction in the incidence rate of complicated ulcers (hazard ratio of 0.50 for COX189 versus NSAIDs) in the COX189 TARGET population of patients not taldng low-dose aspirin. The power will be 95% to detect a 44% reduction in the incidence rate of complicated ulcers (hazard ratio of 0.44 for COX189 versus NSAIDs) in the overall COX189 TARGET population.

By using a generalized Lachin and Foulkes method, a sample size of 18,672 patients (9,336 in both treatment arms) is required to observe 156 serious GI events (56 in the COX189 treatment arm and 100 in the NSAIDs group) in the overall COX189 TARGET population.

No. centers Over 400 sites for each sub study (COX189 0117 & CCOX189A2332)

Target CSOs Worldwide: America/Europe/Asia

1. A pharmaceutical composition for treatment of conditions in mammals which are responsive to COX-2 inhibition which comprises in combination an effective amount of a COX-2 inhibitor and low-dose aspirin, for simultaneous, sequential or separate use.

2. Use of a COX-2 inhibitor for the preparation of a medicament, for use in combination with low-dose aspirin for treatment of conditions in mammals which are responsive to COX-2 inhibition.

3. A method of treating a patient suffering from a condition which is responsive to COX-2 inhibition comprising administering to the patient an effective amount of a COX-2 inhibitor in combination with low-dose aspirin.

4. Use of low-dose aspirin to treat acute coronary ischemic syndrome, thrombosis, thromboembolism, thrombotic occlusion and reocclusion, transient ischemic attack, and first or subsequent thrombotic stroke, in a patient having the condition, when the low-dose aspirin is administered in combination with an effective amount of a COX-2 inhibitor.

5. A compositon according to claim 1 in which the aspirin dose is a dose in the range from about 70 mg down to about 10 mg or less (e.g. at least about 5 mg) per day.

6. A composition according to claim 1 in which the COX-2 inhibitor is a compound or a pharmaceutically acceptable salt thereof, or any hydrate thereof selected from rofecoxib, etoricoxib, celecoxib (Celebrex), valdecoxib, parecoxib, Vioxx, or a 5-alkyl-2-arylaminophenylacetic acid derivative COX-2 inhibitor, e.g. COX189.

7. A composition according to claim 6, in which the COX-2 inhibitor is a compound of formula I 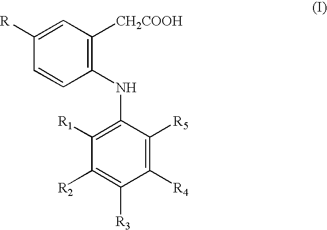
wherein
R is methyl or ethyl;
R₁ is chloro or fluoro;
R₂ is hydrogen or fluoro;
R₃ is hydrogen, fluoro, chloro, methyl, ethyl, methoxy, ethoxy or hydroxy;
R₄ is hydrogen or fluoro; and
R₅ is chloro, fluoro, trifluoromethyl or methyl;
A pharmaceutically acceptable salt thereof; or
pharmaceutically acceptable prodrug esters thereof.

8. A pharmaceutical composition for treatment of conditions in mammals which are responsive to COX-2 inhibition which comprises in combination an effective amount of a compound of formula I or a pharmaceutically acceptable salt or prodrug thereof as defined above and an effective amount of aspirin, for simultaneous, sequential or separate use.

9. Use of a compound of formula I or a pharmaceutically acceptable salt or prodrug thereof for the preparation of a medicament, for use in combination with an effective amount of aspirin for treatment of conditions in mammals which are responsive to COX-2 inhibition.

10. A method of treating a patient suffering from a condition which is responsive to COX-2 inhibition comprising administering to the patient an effective amount of a compound of formula I or a pharmaceutically acceptable salt or prodrug thereof in combination with an effective amount of aspirin.

11. A composition according to claim 8, in which the aspirin dose is a dose in the range from about 10 mg to about 400 mg, more usually from about 75 mg to about 325 mg per day.
